(19) United States
(12) Patent Application Publication
(10) Pub. No.: US 2004/0010213 A1
(43) Pub. Date: Jan. 15, 2004

(54) Stabilizer brace system

(75) Inventors: John H. Gregory, Lake Oswego, OR (US); William G. Brennan, Tualatin, OR (US)

Correspondence Address: Robert D. Varitz, ROBERT D. VARITZ, P.C., 2007 S.E. Grant Street, Portland, OR, 97214, US

(73) Assignee: Mav-Tech Medical L.L.C.

(21) Appl. No.: 10/192,272

(22) Filed: Jul. 10, 2002

(51) Int. Cl. A61F 5/00

(52) U.S. Cl. 602/20

(57) ABSTRACT

An articulated multi-adjustable shoulder, arm, elbow, etc. stabilizer brace system which includes elongate hinged-together stabilizer struts to which are adjustably joined, for effective lateral-positional adjustment, support pads designed to bear against the human anatomy during use.

### BACKGROUND AND SUMMARY OF THE INVENTION

This invention relates, in two different embodiments, to an articulated, multi-adjustable, shoulder, arm, elbow, wrist and hand stabilizer support brace which is useable in the region of the underarm between the upper arm and the upper lateral torso. Specifically the invention relates to an orthotic device which is used, inter alia, for positioning the upper arm at up to approximately a 90° angle (airplane style) from the torso, with or without slight fore or aft angular rotation, following various different kinds of operative shoulder procedures, after which abductive bracing and stabilizing of the arm, etc. to assist in shoulder healing, is desirable. In some instances, only the upper arm is specifically braced. In others the elbow, lower arm, wrist and hand are additionally braced.

There are various surgical procedures involving the shoulder, such as rotator cuff repairs, slap lesions, subacromial decompressions, total shoulder arthroplastics, and others, following which it is desirable, among other things, to stabilize and position the angle which the upper arm extends from the torso. It is, also, sometimes desirable to be able so to stabilize the upper arm while applying and sustaining different levels of fore or aft rotation (in a posterior or anterior sense) relative to the upright axis of the upper torso. There are still further instances where extended, additional bracing is also desirable to support the elbow, the lower arm, the wrist, and the hand.

The present invention provides a novel, highly adjustable othotic device which is very capable of meeting such post-surgical requirements in a structure which is extremely simple and highly adaptable to suit different conditions. According to a preferred embodiment of the invention, and in its simplest form, two elongate rigid struts are hinged together at one set of adjacent ends. These struts carry one or more (each) arcuately shaped, adjustably positional support pads that are adapted to engage, variously, the lateral side of the upper torso, and the underside of the upper arm. Preferably, these support pads are mounted on the mentioned struts in such a fashion that they are adjustable in laterally shiftable, and preferably in a revolvable, sense, and namely, preferably revolvable generally about their respective axes of curvature. To accomplish this preferred form of adjustability, each support pad is equipped with elongate arcuate slots through which screw fasteners, for example, attach them to the struts, whereby they can be loosened and slid (in a revolving manner) back and forth along these slots to achieve an appropriate angular disposition relative to the respective long axes of the struts with which they are specifically associated. Such lateral adjustability offers a high degree of versatility respecting placement of the support pads against the anatomy, without unnecessarily shifting the operative positions of the long axes of the struts to which the supported pads are attached. In other words, an adjustable support pad, according to the invention in its preferred form, can be revolved so as to position it properly for body (torso or arm) engagement, without requiring any substantial repositioning of the long axis of the associated strut whose axis is preferably aligned in a certain fashion with either the torso axis or the upper arm long axis. It should be understood that, fully in accordance with the invention, alternate forms of lateral adjustability, other than revolveability, can be employed for the incorporated support pad, or pads.

According to a modified form of the invention, one of the elongate struts, and specifically that strut whose long axis is intended generally to parallel the long axis of the upper arm during use, carries an orthogonally extendable outrigger structure which is designed to support the elbow, the lower arm, the wrist and the hand in different “flying” (so-called “airplane”) positions relative to the torso.

These special structures, along with their respective features and advantages, as offered by the invention, will be more fully described below.

### DESCRIPTION OF THE DRAWINGS

FIG. 1 is a simplified isometric view of the preferred form of a support brace built in accordance with the present invention.

FIG. 2 illustrates the brace of FIG. 1 placed in one representative stabilizing position and condition relative to the upper lateral torso and the upper arm of a person.

FIGS. 3 and 4 are enlarged, fragmentary, and partially cross-sectional views taken generally along the lines 3-3 and 4-4, respectively, in FIG. 1.

FIG. 5 is a view which is very much like that presented in FIG. 1, except that here what is shown is a modified form of the invention.

FIG. 6 is a view somewhat like that presented in FIG. 3 except that here certain details are shown which help to explain how revolving repositioning of support pads can be accomplished without compromising the most appropriate bracing position for the strut which holds that support pad.

### DETAILED DESCRIPTION OF THE INVENTION

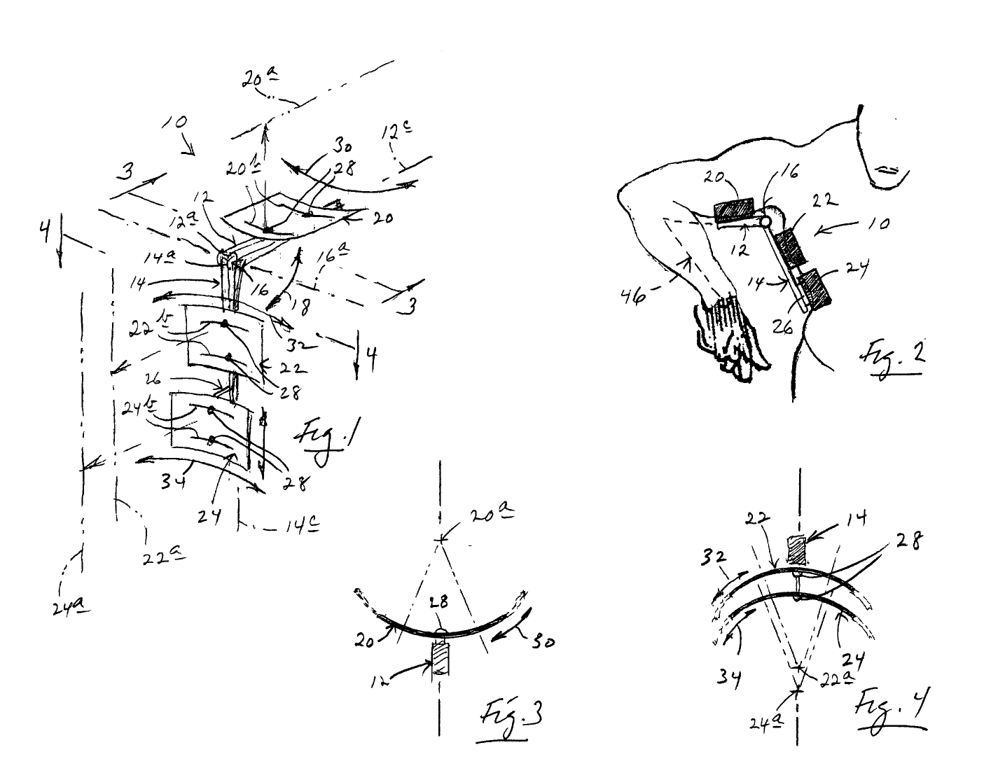

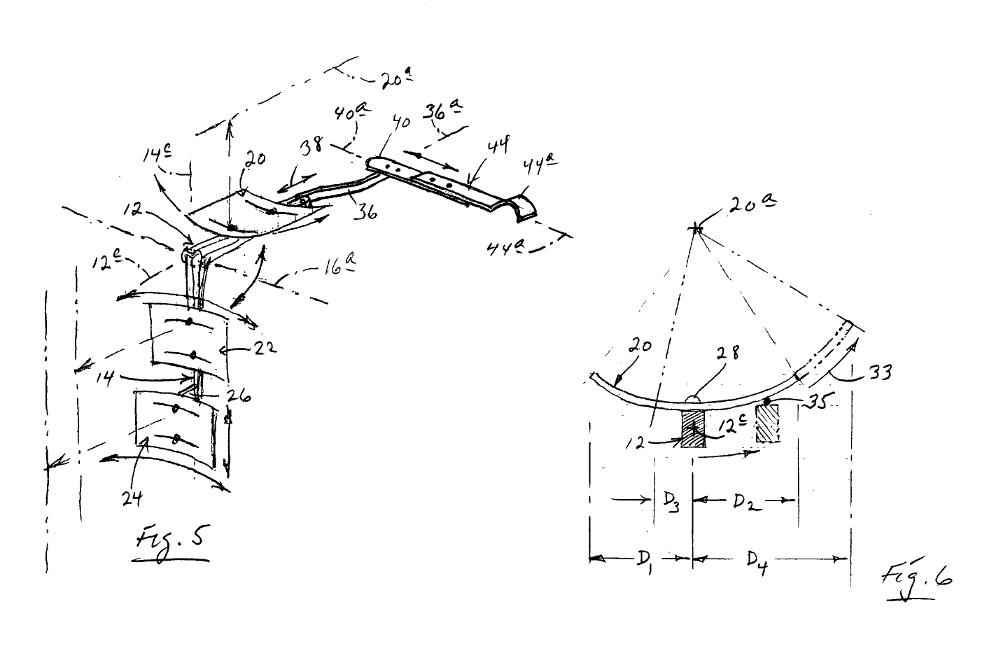

Turning now to the drawings, and referring first of all to FIGS. 1-4 collectively, indicated generally at 10 is an articulated, multi-adjustable support brace which is constructed in accordance with a preferred form of the present invention. Brace 10 includes two elongate struts 12, 14 having one set of adjacent ends 12a, 14a, respectively, which are hinged together, or pivoted, through an articulation pivot connection (hinge) shown at 16. The pivot axis (also called a hinge axis) provided by connection 16 is shown at 16a. Strut 12 is the shorter one of these two struts, and specifically is the strut which, during use of the brace, will extend along the underside of the upper arm in the region near the underarm. Strut 14, the longer strut, will extend, during use of the brace, generally in an upright fashion along, and somewhat paralleling, the long axis of the upper torso. Struts 12, 14 have respective long axes shown at 12c, 14c in FIG. 1. With the brace in place during use (as shown in FIG. 2) hinge 16 is disposed generally centrally within the region of the underarm.

While different specific hinge constructions can be employed, connection 16 herein allows for releasably lockable angular positioning of the long axes of struts 12, 14 relative to one another, generally throughout an angular range of about 90°. The possibility for such angular adjustability is represented by double-ended curved arrow 18 seen in FIG. 1. Through appropriate releasable locking hardware, such as loosenable and tightenable screws (not shown) that are present within hinge connection 16, an angularly locked condition between the two struts can be released to permit adjustment, and then relocked to position the two struts appropriately angularly for each particular bracing use.

According to the invention, several arcuate support pads are provided in brace 10, and three such pads are shown at 20, 22, 24 in FIGS. 1-4, inclusive. Pad 20 is mounted as will shortly be described, on strut 12. Pads 22, 24 are mounted on strut 14. Pads 20, 22 are essentially mounted directly on struts 12, 14, respectively, and pad 24 is mounted near the distal extremity (relative to hinge connection 16) of strut 14 through a spacer, or standoff, structure 26.

The exact arcuate curvatures of these three pads, which can be the same or different, are not especially important. Nor is it important that the curvatures of the pads be truly circular in nature. However, it is convenient to think of these pads as curving about respective axes of curvature, and such axes for pads 20, 22, 24 are shown generally at 20a, 22a, 24a, respectively. Because of the standoff mounting provided as just mentioned for pad 24, axis of curvature 24a is somewhat more distant from the long axis of strut 14 than is axis 22a (see especially FIG. 1).

According to the preferred form of the invention now being described, pads 20, is 22, 24 are provided with pairs of spaced, generally parallel, arcuate slots 20b, 22b, 24b, respectively. These slots receive loosenable/tightenable anchoring devices (mounting structure), such as the screws shown at 28. By loosening the specific screws which anchor each individual pad in place, the associated pad (or pads) can be rocked in a revolving kind of fashion, generally along arcuate paths which curve around the respective axes of curvatures of the pad(s). Thus, one will see that pad 20 can be laterally adjusted in a revolving manner as indicated by arrow 30, pad 22 as indicated by arrow 32, and pad 24 as indicated by arrow 34. One will observe that such lateral adjustability which is furnished for these pads allows them to be positioned in a manner so as to engage the torso or the upper arm appropriately without unnecessarily displacing the spatial locations, relative to the arm and torso, of the long axes of struts 12, 14. Such adjustability thus provides use-accommodation, along the lines just suggested, without compromising appropriate bracing-support positioning for the articulated struts in device 10.

Not specifically illustrated in the drawings is the possibility of providing for “longitudinal” adjustability of the pads along the lengths of their respective associated struts.

Digressing for a moment here to FIG. 6. As has been mentioned, one of the interesting features of the present invention is that the support pads that are provided on the struts for bearing against different parts of the body can be adjusted so that they can engage the body at different locations without disrupting, necessarily, the most appropriate bracing position for the associate strut. In FIG. 6 support pad 20 is shown in solid lines substantially laterally centered relative to strut 12, and more specifically, relative to the long axis of this strut which extends essentially normal to the plane of FIG. 6. In this laterally centered position, the opposite lateral edges, so-to-speak, of pad 20 lie at the distances shown at D1 and D2 in FIG. 6.

If it were desired to produce a lateral shift, and a kind of re-angulation of support pad 20, in order to accommodate a particular bracing situation, according to the invention, attaching screws 28 are loosened, and the support pad is adjusted so-as-to shift it laterally in the appropriate direction. Such a shift is generally illustrated in FIG. 6 by the dash-double-dot lines therein, and by curved arrow 33. Very specifically, from the point of view taken in FIG. 6, pad 20 is revolved and shifted generally to the right in FIG. 6, whereby it subtends an angle relative to its axis of curvature 28a which lies between the two dash-double-dot lines that extend from this axis. With this shift having taken place, one will observe that pad 20 has perhaps been shifted quite appropriately to the position for different positional engagement on the body, yet strut 12 has remained essentially in the same position unmoved in the field of view of FIG. 6.

By way of contrast, if such an adjustment were only permitted by producing angulation of the support pad so that it would properly engage the anatomy, and were that angulation and adjustment permitted by, for example, a pivot connection rather than a sliding/revolving type connection, the lateral edges of the support pad would not shift and change effectively their distances from strut 12, and the strut would need to be repositioned in order to accommodate the adjustment made in the support pad. Strut 12 in FIG. 6 is shown in solid lines in the position first described for it. In dash-double-dot lines in FIG. 6, however, strut 12 is shown in the shifted position which it would need to occupy if angulation and adjustment of the support pad could only take place through a pivot connection, such as the connection shown at 35 in FIG. 6. Accordingly, if the most appropriate bracing position for strut 12 were that in which it is pictured in solid lines in FIG. 6, an adjustment requiring shifting of this strut, as is illustrated in FIG. 6, would thereby move the strut out of and away from the most appropriate bracing position.

It is this lateral, and preferably revolving-type, adjustability, provided according to the present invention for the support pads, that permits a significant amount of angular and positional adjustment to be introduced to the support pads without compromising the most appropriate support and bracing position for the associated struts.

Thinking of what has just been described in slightly different terms, whereas a pivot-type connection between a strut and a support pad would result in adjustment of the pad not producing any change in the relative lateral distances that exist between the opposite lateral edges of the pad and the associated strut, revolving type adjustments as permitted by the invention indeed produce a definite change in lateral distances between the opposite lateral edges of the pad and the supporting strut. And so, one can see in FIG. 6 how, with adjustment of pad 20 to the dashed-double-dot line position under circumstances of strut 12 remaining unmoved, the lateral distances to opposite lateral sides of support pad 20 relative to strut 12 change dramatically to those spacings which are shown at D3 and D4.

This important feature of mountings provided for support pads herein plays an important role in enhancing utility of the brace structure offered by the present invention.

FIG. 2 in the drawings, as was mentioned earlier, generally pictures device 10 in place under the (right) lateral upper torso and (right) upper arm of a person. Here, one can see how the standoff (or offset) mounting furnished for pad 24, relative to the position of pad 22, accommodates fitment of the brace in place against a tapering torso, while keeping the disposition of strut 14 most appropriately positioned along the side of the torso. In a real bracing installation, device 10 would not simply be placed in a freeform fashion as pictured in FIG. 2, but rather would be appropriately bound in place through other, typically soft, structure. Also, the concave contacting faces, so-to-speak, of the arcuate pads would typically be provided with some form of soft cushioning (not shown herein).

Thus one can see that device as so far described is a very versatile device in terms of offering highly adaptable and repositionable bracing support in the region of the anatomy so far discussed. Modifications can certainly be made in the exact sizes and curvatures of the support pads, and it is certainly also possible to employ a modified form of device 10 which might use only a single torso-contacting, curved support pad. By making appropriate revolving (or other lateral) repositionings of the pads, many specific bracing conditions can be accommodated, including a certain amount of fore or aft rotation of the arm relative to the torso. Such can be done in a manner which retains proper, and the most effective, positioning of struts 12, 14.

Turning attention now to FIG. 5 in the drawings, here there is shown a modified form of the invention in which what is referred herein as an othogonally adjustable outrigger structure is fastened appropriately to strut 12. This outrigger structure is provided in order to furnish support for the elbow, the lower arm, the wrist and the hand during a bracing operation. Thus, releasably and adjustably anchored to, and extending longitudinally outwardly from, the distal end of strut 12 (relative to hinge connection 16) is a first strut extension 36 which, as indicated by double-headed arrow 38, can be affixed to strut 12 in different longitudinally extending conditions. The exact shape of extension 36 is a matter of choice. Extension 36 has a long axis shown at 36a.

Mounted adjacent the outer end of extension 36 is an elongate extension 40 which has a long axis 40a that, in general terms, extends orthogonally relative to the long axis of extension 36. Appropriate releasable and tightenable affixing structure is employed in the region of connection between extensions 36, 40 to allow extension 40 to be adjusted reversibly and longitudinally as is generally indicated by double-headed linear arrow 42. Extensions 36, 40 are referred to collectively herein as plural-part, right-angle structure.

Finally pictured in FIG. 5 is yet another extension 44 which has a long axis 44a, and which is fastened in a longitudinally paralleling fashion with respect to extension 40 for releasable and lockable adjustment back and forth, as is also generally pictured by previously mentioned double-headed arrow 42. The outer extremity of extension 44 is formed with an uprising arcuate portion 44a which has a kind of inverted cup shape that is intended to receive the underside of a supported hand. This structure is also referred the herein as palm-of-hand support structure.

The extension structure assembly which has just been described, and which is illustrated in FIG. 5, is referred to as an orthogonally adjustable structure inasmuch as it can be moved and adjusted for length and positioning as illustrated by arrows 38, 42 which are generally orthogonal relative to one another. While the device of FIG. 5 is not specifically illustrated supporting the human arm structure, the extension structure which has just been described, when used, generally follows the angular dashed lines that appear at 46 in FIG. 2.

Thus, while a preferred embodiment, and certain modifications, of the present invention have been shown and described herein, in is appreciated that other variations and modifications may be made without departing from the spirit of the invention.

I claim:

1. An articulated, multi-adjustable support brace usable in the region of the underarm, between the upper arm and the upper lateral torso comprising
first and second elongate struts, each having a long axis, and an end pivoted to an adjacent end in the other strut, said struts being swingably adjustable respecting the pivot axis between them to establish different selected relative angular orientations of the struts with respect to one another, and
an arcuate support pad adjustably mounted through mounting structure on at least one of said struts, curving about an effective axis of curvature which is displaced from and substantially parallel to the long axis of said at least one strut, said mounting structure cooperating with said pad to permit positional revolution of the pad relative to the at least one strut generally about said pad's said effective axis of curvature.

2. The brace of claim 1 which further includes at least one other support pad mounted through mounting structure on the other arm.

3. The brace of claim 2 which further comprises at least one additional support pad on one of the two struts.

4. The brace of claim 3, wherein, regarding the strut which carries the two support pads, these pads are spaced along the length of the strut, with the support pad which is the more distant from the pivot axis for the struts being positioned further from the long axis of its associated strut than is the other support pad which is also associated with that same strut.

5. The brace of claim 1, wherein said pivoted struts are releasably lockable against movement relative to one another.

6. The brace of claim 1, wherein said support pad is releasably lockable in position relative to said at least one strut.

7. The brace of claim 2, wherein said at least one other support pad is releasably lockable against movement relative to said other strut.

8. The brace of claim 3, wherein all support pads are releasably lockable against movements relative to their respective associated struts.

9. The brace of claim I which further includes an elongate lower-arm support extension adjustably joined to one of said struts, and having a long axis which extends at an angle relative to the long axis of said one strut.

10. The brace of claim 9, wherein said extension has an outer distal end which carries an arcuate palm-of-hand support structure.

11. The brace of claim 9, wherein the structure which adjustably joins said support extension to said one strut takes the form of a plural-part, right-angle structure which is orthogonally length-adjustable.

12. An articulated multi-adjustable support brace useable in the region of the underarm, between the upper arm and the upper lateral torso, said brace comprising
first and second elongate struts, each having a long axis, and an end pivoted to an adjacent end in the other strut, said struts being swingably adjustable respecting the pivot axis between them to establish different connected relative angular orientation of the struts with respect to one another, and
an arcuate support pad adjustably mounted through mounting structure on at least on of said struts, laterally adjustable relative to the long axis of its associated strut by positioning the lateral margins of the pad at selectively different instances from the strut axis.
